(19) United States
(12) Patent Application Publication
(10) Pub. No.: US 2019/0282714 A1
(43) Pub. Date: Sep. 19, 2019

(54) RADIOLIGANDS FOR IMAGING THE IDO1 ENZYME

(71) Applicant: BRISTOL-MYERS SQUIBB COMPANY, Princeton, PA (US)

(72) Inventors: David J. Donnelly, Stockton, NJ (US); Erin Lee Cole, Philadelphia, PA (US); Richard Charles Burrell, East Haddam, CT (US); Wesley A. Turley, Hamilton Township, NJ (US); Alban J. Allentoff, Flemington, NJ (US); Michael Arthur Wallace, Ringoes, NJ (US); James Aaron Balog, Lambertville, NJ (US); Audris Huang, New Hope, PA (US); Mette Skinbjerg, Morrisville, PA (US)

(21) Appl. No.: 16/318,209

(22) PCT Filed: Jul. 18, 2017
(86) PCT No.: PCT/US2017/042510

§ 371 Date: Jan. 16, 2019

Related U.S. Application Data
(60) Provisional application No. 62/364,020, filed on Jul. 19, 2016.

(51) Int. Cl. A61K 51/04; C07D 215/18; C07B 59/00

(52) U.S. Cl. CPC A61K 51/0455; C07B 59/002; C07D 215/18

(57) ABSTRACT

The present invention relates to radiolabeled IDO1 inhibitors or pharmaceutically acceptable salts thereof which are useful for the quantitative imaging of IDO enzymes in mammals.

### CROSS-REFERENCE TO RELATED APPLICATIONS

This application claims priority to U.S. Provisional Application Ser. No. 62/364,020, filed Jul. 19, 2016, the entire content of which is incorporated herein by reference.

### FIELD OF THE INVENTION

The invention relates to novel radiolabeled IDO1 inhibitors and their use in labeling and diagnostic imaging of IDO enzymes in mammals.

### BACKGROUND OF THE INVENTION

Positron emission tomography (PET) is a non-invasive imaging technique that can provide functional information about biological processes in living subjects. The ability to image and monitor in vivo molecular events, are great value to gain insight into biochemical and physiological processes in living organisms. This in turn is essential for the development of novel approaches for the treatment of diseases, early detection of disease and for the design of new drugs. PET relies on the design and synthesis of molecules labeled with positron-emitting radioisotope. These molecules are known as radiotracers or radioligands. For PET imaging, the most commonly used positron emitting (PET) radionuclides are; ¹¹C, ¹⁸F, ¹⁵O and ¹³N, all of which are cyclotron produced, and have half lives of 20, 110, 2 and 10 minutes, respectively. After being radiolabeled with a positron emitting radionuclide, these PET radioligands are administered to mammals, typically by intravenous (i.v.) injection. Once inside the body, as the radioligand decays it emits a positron that travels a small distance until it combines with an electron. An event known as an annihilation event then occurs, which generates two collinear photons with an energy of 511 keV each. Using a PET imaging scanner which is capable of detecting the gamma radiation emitted from the radioligand, planar and tomographic images reveal distribution of the radiotracer as a function of time. PET radioligands provide useful in-vivo information around target engagement and dose dependent binding site occupancy for receptors and enzymes.

Indoleamine 2,3-dioxygenase (IDO; also known as IDO1) is an IFN-γ target gene that plays a role in immunomodulation. IDO1 is an oxidoreductase and one of two enzymes that catalyze the first and rate-limiting step in the conversion of tryptophan to N-formyl-kynurenine. It exists as a 41 kD monomer that is found in several cell populations, including immune cells, endothelial cells, and fibroblasts. IDO1 is relatively well-conserved between species, with mouse and human sharing 63% sequence identity at the amino acid level. Data derived from its crystal structure and site-directed mutagenesis show that both substrate binding and the relationship between the substrate and iron-bound dioxygenase are necessary for activity. A homolog to IDO1 (IDO2) has been identified that shares 44% amino acid sequence homology with IDO, but its function is largely distinct from that of IDO1. (See, e.g., Serafini, P. et al., Semin. Cancer Biol., 16(1):53-65 (February 2006) and Ball, H. J. et al., Gene, 396(1):203-213 (Jul. 1, 2007)).

IDO1 plays a major role in immune regulation, and its immunosuppressive function manifests in several manners. Importantly, IDO1 regulates immunity at the T cell level, and a nexus exists between IDO1 and cytokine production. In addition, tumors frequently manipulate immune function by upregulation of IDO1. Thus, modulation of IDO1 can have a therapeutic impact on a number of diseases, disorders and conditions.

A pathophysiological link exists between IDO1 and cancer. Disruption of immune homeostasis is intimately involved with tumor growth and progression, and the production of IDO1 in the tumor microenvironment appears to aid in tumor growth and metastasis. Moreover, increased levels of IDO1 activity are associated with a variety of different tumors (Brandacher, G. et al., Clin. Cancer Res., 12(4):1144-1151 (Feb. 15, 2006)).

Treatment of cancer commonly entails surgical resection followed by chemotherapy and radiotherapy. The standard treatment regimens show highly variable degrees of long-term success because of the ability of tumor cells to essentially escape by regenerating primary tumor growth and, often more importantly, seeding distant metastasis. Recent advances in the treatment of cancer and cancer-related diseases, disorders and conditions comprise the use of combination therapy incorporating immunotherapy with more traditional chemotherapy and radiotherapy. Under most scenarios, immunotherapy is associated with less toxicity than traditional chemotherapy because it utilizes the patient's own immune system to identify and eliminate tumor cells.

In addition to cancer, IDO1 has been implicated in, among other conditions, immunosuppression, chronic infections, and autoimmune diseases or disorders (e.g., rheumatoid arthritis). Thus, suppression of tryptophan degradation by inhibition of IDO1 activity has tremendous therapeutic value. Moreover, inhibitors of IDO1 can be used to enhance T cell activation when the T cells are suppressed by pregnancy, malignancy, or a virus (e.g., HIV). Although their roles are not as well defined, IDO1 inhibitors may also find use in the treatment of patients with neurological or neuropsychiatric diseases or disorders (e.g., depression).

Use of a specific PET radioligand having high affinity for IDO1 in conjunction with supporting imaging technology may provide a method for clinical evolution around both target engagement and dose/occupancy relationships of IDO1 inhibitors in tissues that express IDO1 such as the lung, gut, and dendritic cells of the immune system. The invention described herein relates to radiolabeled IDO1 inhibitors that would be useful for the exploratory and diagnostic imaging applications, both in-vitro and in-vivo, and for competition studies using radiolabeled and unlabeled IDO1 inhibitors.

### BRIEF DESCRIPTION OF THE DISCLOSURE

The present disclosure is based, in part, on the appreciation that radiolabeled IDO1 inhibitors are useful in the detection and/or quantification and/or imaging of IDO1 enzymes and/or IDO1 expression and/or affinity of a compound for occupying the binding site of the IDO1 enzyme in tissue of a mammalian species. It has been found that radiolabeled IDO1 inhibitors, when administered to a mammalian species, build up at or occupy the active site on the IDO1 enzyme and can be detected through imaging techniques, thereby providing valuable diagnostic markers for presence of IDO1 proteins, affinity of a compound for occupying the active site of an IDO1 enzyme, and clinical evaluation and dose selection of IDO1 inhibitors. In addition, the radiolabeled IDO1 inhibitors disclosed herein can be used as a research tool to study the interaction of unlabeled IDO1 inhibitors with IDO1 enzymes in vivo via competition between the unlabeled drug and the radiolabeled drug for binding to the enzyme. These types of studies are useful in determining the relationship between IDO1 enzyme active site occupancy and dose of unlabeled IDO1 inhibitor, as well as for studying the duration of blockade of the enzyme by various doses of unlabeled IDO1 inhibitors.

As a clinical tool, the radiolabeled IDO1 inhibitor can be used to help define clinically efficacious doses of IDO1 inhibitors. In animal experiments, the radiolabeled IDO1 inhibitor can be used to provide information that is useful for choosing between potential drug candidates for selection for clinical development. The radiolabeled IDO1 inhibitor can also be used to study the regional distribution and concentration of IDO1 enzymes in living tissues. They can be used to study disease or pharmacologically related changes in IDO1 enzyme concentrations.

According to the present invention, the following compound of Formula I is provided:

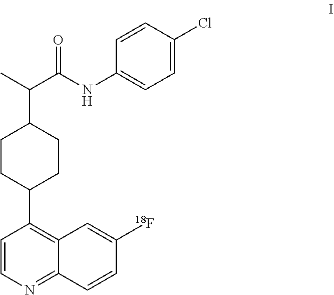 [SMILES: CC(C(=O)Nc1ccc(Cl)cc1)C1CCC(c2ccnc3ccc([18F])cc23)CC1]

including pharmaceutically acceptable salts thereof and stereoisomers such as:

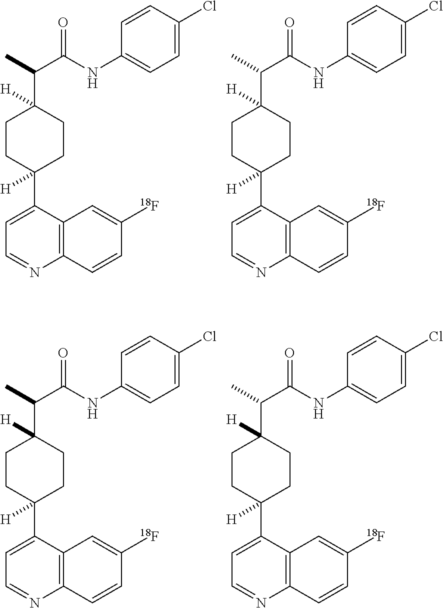 [SMILES: C[C@@H](C(=O)Nc1ccc(Cl)cc1)[C@H]1CC[C@@H](c2ccnc3ccc([18F])cc32)CC1 ; C[C@H](C(=O)Nc1ccc(Cl)cc1)[C@H]1CC[C@@H](c2ccnc3ccc([18F])cc32)CC1 ; C[C@@H](C(=O)Nc1ccc(Cl)cc1)[C@H]1CC[C@H](c2ccnc3ccc([18F])cc32)CC1 ; C[C@H](C(=O)Nc1ccc(Cl)cc1)[C@H]1CC[C@H](c2ccnc3ccc([18F])cc32)CC1]

According to one embodiment of the present invention, pharmaceutical compositions are provided, comprising a diagnostically effective amount of the radiolabeled compound of Formula I together with a pharmaceutically acceptable carrier therefor.

The present invention also provides a method for the in vivo imaging of mammalian tissues of known IDO1 expression to detect cancer cells, such method comprising the steps of:

(a) administering the radiolabeled compound of Formula I as described herein to a subject; and

(b) imaging in vivo the distribution of the radiolabeled compound by positron emission tomography (PET) scanning.

According to one embodiment of the present invention, a method for screening a non-radiolabeled compound to determine its affinity for occupying the active site of an IDO1 enzyme in mammalian tissue is provided comprising the steps of:

(a) administering the radiolabeled compound of Formula I to a subject;

(b) imaging in vivo tissues of known IDO1 expression by positron emission tomography (PET) to determine a baseline uptake of the radiolabeled compound;

(c) administering the non-radiolabeled compound to the subject;

(d) administering a second dose of the radiolabeled compound of Formula I to the subject;

(e) imaging in vivo the distribution of the radiolabeled compound of Formula I in tissues that express the IDO1 enzyme;

(f) comparing the signal from PET scan data at baseline within the tissue that expresses IDO1 to PET scan data retrieved after administering the non-radiolabeled compound within the tissue that expresses IDO1.

According to one embodiment of the present invention, a method for monitoring the treatment of a cancer patient who is being treated with an IDO1 inhibitor is provided comprising the steps of:

(a) administering to the patient the radiolabeled compound of Formula I,

(b) obtaining an image of tissues in the patient that express IDO1 by positron emission tomography (PET); and

(c) detecting to what degree said radiolabeled IDO1 inhibitor occupies the active site of the IDO1 enzyme.

According to one embodiment of the present invention, a method for tissue imaging is provided comprising the steps of contacting a tissue that contains IDO1 enzymes with the radiolabeled compound of Formula I, as described herein, and detecting the radiolabeled compound using positron emission tomography (PET) imaging, wherein said detection can be done in vitro or in vivo.

According to one embodiment of the present invention, a method for diagnosing the presence of a disease in a subject is provided, comprising,

(a) administering to a subject the radiolabeled compound of Formula I which binds to the IDO1 enzyme associated with the presence of the disease; and

(b) obtaining a radio-image of at least a portion of the subject to detect the presence or absence of the radiolabeled compound; wherein the presence and location of the radiolabeled compound above background is indicative of the presence or absence of the disease.

According to one embodiment of the present invention, a method for quantifying diseased cells or tissue is provided, comprising;

(a) administering to a subject having diseased cells or tissues the radiolabeled compound of Formula I, which binds to the IDO1 enzyme located within the diseased cells or tissues; and

(b) detecting radioactive emissions of the radiolabeled compound in the diseased cells or tissues, wherein the level and distribution of the radioactive emissions in the diseased cells or tissues is a quantitative measure of the diseased cells or tissues.

### BRIEF DESCRIPTION OF THE DRAWINGS

FIG. 1 is a schematic of an automated synthesis of [¹⁸F](R)-N-(4-chlorophenyl)-2-(1S,4S)-4-(6-fluoroquinolin-4-yl)cyclohexyl)propanamide using a Synthera synthesis unit and custom purification system.

FIG. 2 is a radiotracer uptake bar graph showing the following: A) Tracer uptake in M109 tumors after 4-5 days of treatment with vehicle (n=10) or non-radioactive (R)-N-(4-chlorophenyl)-2-((1S,4S)-4-(6-fluoroquinolin-4-yl)cyclohexyl)propanamide; 6 mg/kg (n=12), 60 mg/kg (n=12) and 150 mg/kg (n=11). Administration of (R)-N-(4-chlorophenyl)-2-((1S,4S)-4-(6-fluoroquinolin-4-yl)cyclohexyl)propanamide produced a dose-dependent displacement of the tracer compared to vehicle. The dotted line represents average tracer uptake in muscle tissue. B) Tracer uptake in muscle reference tissue after 4-5 days of treatment with vehicle (n=10) or of non-radioactive (R)-N-(4-chlorophenyl)-2-((1S,4S)-4-(6-fluoroquinolin-4-yl)cyclohexyl)propanamide(R)-N-(4-chlorophenyl)-2-((1S,4S)-4-(6-fluoroquinolin-4-yl)cyclohexyl)propanamide; 6 mg/kg (n=12), 60 mg/kg (n=12) and 150 mg/kg (n=11). Administration of (R)-N-(4-chlorophenyl)-2-((1S,4S)-4-(6-fluoroquinolin-4-yl)cyclohexyl)propanamide had no effect on the uptake in muscle tissue. C) Consistent with the imaging results, 5-6 days of treatment with vehicle (n=7) or non-radioactive (R)-N-(4-chlorophenyl)-2-((1S,4S)-4-(6-fluoroquinolin-4-yl)cyclohexyl)propanamide; 6 mg/kg (n=7), 60 mg/kg (n=8) and 150 mg/kg (n=8) produced a dose-dependent inhibition of the Kynurenine pathway measured as the ratio of Kynurenine to tryptophan. D) 5-6 days of treatment with either 6 mg/kg (n=7), 60 mg/kg (n=8) or 150 mg/kg (n=8) of non-radioactive (R)-N-(4-chlorophenyl)-2-((1S,4S)-4-(6-fluoroquinolin-4-yl)cyclohexyl)propanamide resulted in a dose-dependent increase in serum concentration of (R)-N-(4-chlorophenyl)-2-((1S,4S)-4-(6-fluoroquinolin-4-yl)cyclohexyl)propanamide.

FIG. 3 is a radiotracer uptake bar graph showing tracer uptake in M109 tumors before (BL, Solid bars) and after treatment with vehicle (n=4) or non-radioactive (R)-N-(4-chlorophenyl)-2-((1S,4S)-4-(6-fluoroquinolin-4-yl)cyclohexyl)propanamide; 6 mg/kg (n=4), 60 mg/kg (n=4) and 150 mg/kg (n=4) (treat, Striped bars). Before treatment was administered (baseline), tracer uptake did not differ between groups. After treatment, there was no change in tracer uptake in the vehicle group, but administration of (R)-N-(4-chlorophenyl)-2-((1S,4S)-4-(6-fluoroquinolin-4-yl)cyclohexyl)propanamide produced a dose-dependent displacement of the tracer. The dotted line represents average tracer uptake in muscle reference tissue.

FIG. 4 is a radiotracer uptake bar graph showing tracer uptake was increased in M109 tumors (n=10) with high IDO1 expression compared to CT26 tumors (n=10) with low IDO1 expression.

FIG. 5 are MRI and PET images of a Cynomolgus monkey imaged with ¹⁸F-(R)-N-(4-chlorophenyl)-2-((1S,4S)-4-(6-fluoroquinolin-4-yl)cyclohexyl)propanamide were generated. A total of five consecutive full body images were obtained to evaluate tracer kinetics and biodistribution over time. The tracer accumulated in expected clearance organs such as liver and gallbladder while little to no background was observed in the remainder body.

### DETAILED DESCRIPTION OF THE INVENTION

In a first embodiment of the present invention, a compound of the following Formula I or a pharmaceutically acceptable salt thereof is provided:

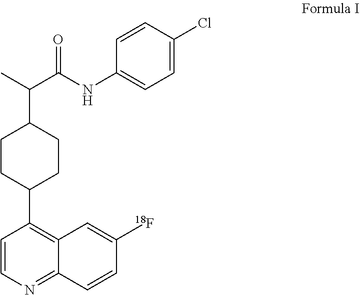 [SMILES: CC(C(=O)Nc1ccc(Cl)cc1)C1CCC(c2ccnc3ccc([18F])cc23)CC1]

Stereoisomers of Formula I are also included in the scope of the invention and include, for example, the following:

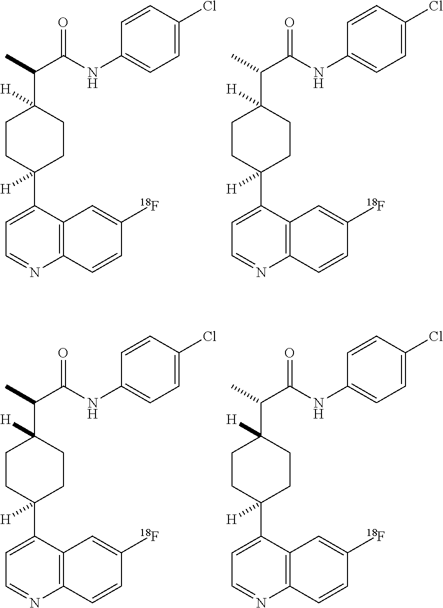 [SMILES: C[C@@H](C(=O)Nc1ccc(Cl)cc1)[C@H]1CC[C@@H](c2ccnc3ccc([18F])cc32)CC1 ; C[C@H](C(=O)Nc1ccc(Cl)cc1)[C@H]1CC[C@@H](c2ccnc3ccc([18F])cc32)CC1 ; C[C@@H](C(=O)Nc1ccc(Cl)cc1)[C@H]1CC[C@H](c2ccnc3ccc([18F])cc32)CC1 ; C[C@H](C(=O)Nc1ccc(Cl)cc1)[C@H]1CC[C@H](c2ccnc3ccc([18F])cc32)CC1]

The compound of Formula I is a radiolabeled IDO1 inhibitor which is useful as a positron emitting molecule having IDO1 enzyme affinity.

According to one embodiment of the present invention, the present disclosure provides a diagnostic composition for imaging IDO1 enzymes which includes a radiolabeled IDO1 inhibitor and a pharmaceutically acceptable carrier. In yet another embodiment, the present disclosure provides a method of autoradiography of mammalian tissues of known IDO1 expression, comprising the steps of administering a radiolabeled IDO1 inhibitor to a patient, obtaining an image of the tissues by positron emission tomography, and detecting the radiolabeled compound in the tissues to determine IDO1 target engagement and occupancy of the active site of the IDO1 enzyme.

Radiolabeled IDO1 inhibitors, when labeled with the appropriate radionuclide, are potentially useful for a variety of in vitro and/or in vivo imaging applications, including diagnostic imaging, basic research, and radiotherapeutic applications. Specific examples of possible diagnostic imaging and radiotherapeutic applications include determining the location of, the relative activity of and/or quantification of IDO1 enzymes; radioimmunoassay of IDO1 inhibitors; and autoradiography to determine the distribution of IDO1 enzymes in a patient or an organ or tissue sample thereof.

In particular, the instant radiolabeled IDO1 inhibitor is useful for positron emission tomographic (PET) imaging of IDO1 enzymes in the lung, gut, and dendritic cells of the immune system or other organs of living humans and experimental animals. The radiolabeled IDO1 inhibitor of the present invention may be used as research tool to study the interaction of unlabeled IDO1 inhibitors with IDO1 enzymes in vivo via competition between the unlabeled drug and the radiolabeled compound for binding to the enzyme. These types of studies are useful for determining the relationship between IDO1 enzyme occupancy and dose of unlabeled IDO1 inhibitor, as well as for studying the duration of blockade of the enzyme by various doses of the unlabeled IDO1 inhibitor. As a clinical tool, the radiolabeled IDO1 inhibitor may be used to help define a clinically efficacious dose of an unlabeled IDO1 inhibitor. In animal experiments, the radiolabeled IDO1 inhibitor can be used to provide information that is useful for choosing between potential drug candidates for selection for clinical development. The IDO1 inhibitors may also be used to study the regional distribution and concentration of IDO1 in the lung, gut, and dendritic cells of the immune system and other IDO1-expressing tissues, and other organs of living experimental animals and in tissue samples. The radiolabeled IDO1 inhibitors may also be used to study disease or pharmacologically related changes in IDO1 enzyme concentrations.

For example, positron emission tomography (PET) tracers such as the radiolabeled IDO1 inhibitor of the present invention can be used with currently available PET technology to obtain the following information: relationship between level of enzyme binding site occupancy by candidate IDO1 inhibitors and clinical efficacy in patients; dose selection for clinical trials of IDO1 inhibitors prior to initiation of long term clinical studies; comparative potencies of structurally novel IDO1 inhibitors; investigating the influence of IDO1 inhibitors on in vivo transporter affinity and density during the treatment of clinical targets with IDO1 inhibitors; changes in the density and distribution of IDO1 during effective and ineffective treatment of cancer or other IDO1 mediated diseases.

The present radiolabeled IDO1 inhibitor has utility in imaging IDO1 enzymes or for diagnostic imaging with respect to a variety of disorders associated with IDO1 expression.

For the use of the instant compounds as exploratory or diagnostic imaging agents, the radiolabeled compound may be administered to mammals, preferably humans, in a pharmaceutical composition either alone or, preferably, in combination with pharmaceutically acceptable carriers or diluents, optionally with known adjuvants, such as alum, in a pharmaceutical composition, according to standard pharmaceutical practice. Such compositions can be administered orally or parenterally, including the intravenous, intramuscular, intraperitoneal, subcutaneous, rectal and topical routes of administration. Preferably, administration is intravenous. The inhibitor is a radiotracer labeled with a short-lived, positron emitting radionuclide and thus is generally administered via intravenous injection within less than one hour of synthesis. This is necessary because of the short half-life of the radionuclide involved.

An appropriate dosage level for the unlabeled IDO1 inhibitor ranges from between 1 mg to 1500 mg and is preferably from 25 mg to 800 mg daily. When the present radiolabeled IDO1 inhibitor is administered to a human subject, the amount required for imaging will normally be determined by the prescribing physician with the dosage generally varying according to the quantity of emission from the radionuclide. However, in most instances, an effective amount will be the amount of compound sufficient to produce emissions in the range of from about 1-5 mCi.

In one exemplary application, administration occurs in an amount between 0.5-20 mCi of total radioactivity injected into a patient depending upon the subjects body weight. The upper limit is set by the dosimetry of the radiolabeled molecule in either rodent or non-human primate.

The following illustrative procedure may be utilized when performing PET imaging studies on patients in the clinic. The patient is pre-medicated with unlabeled IDO1 inhibitor some time prior to the day of the experiment and is fasted for at least 12 hours allowing water intake ad libitum. A 20 G two-inch venous catheter is inserted into the contralateral ulnar vein for radiotracer administration. Administration of the PET tracer is often timed to coincide with time of maximum (Tₘₐₓ) or minimum (Tₘᵢₙ) of IDO1 inhibitor concentration in the blood.

The patient is positioned in the PET camera and a tracer dose of the PET tracer of radiolabeled IDO1 inhibitor such as Example 5A (<20 mCi) is administered via i.v. catheter. Either arterial or venous blood samples are taken at appropriate time intervals throughout the PET scan in order to analyze and quantitate the fraction of unmetabolized PET tracer in plasma. Images are acquired for up to 120 min. Within ten minutes of the injection of radiotracer and at the end of the imaging session, 1 ml blood samples are obtained for determining the plasma concentration of any unlabeled IDO1 inhibitor which may have been administered before the PET tracer.

Tomographic images are obtained through image reconstruction. For determining the distribution of radiotracer, regions of interest (ROIs) are drawn on the reconstructed image including, but not limited to, the lung, gut, and dendritic cells of the immune system as well as other IDO1 expressing tissues or other organs. Radiotracer uptakes over time in these regions are used to generate time activity curves (TAC) obtained in the absence of any intervention or in the presence of the unlabeled IDO1 inhibitor at the various dosing paradigms examined. Data are expressed as radioactivity per unit time per unit volume (μCi/cc/mCi injected dose). TAC data are processed with various methods well-known in the field to yield quantitative parameters, such as Binding Potential (BP) or Volume of Distribution (V_{T}), that are proportional to the density of unoccupied IDO1 binding site. Inhibition of the IDO1 enzyme is then calculated based on the change of BP or V_{T} by equilibrium analysis in the presence of IDO1 inhibitors at the various dosing paradigms as compared to the BP or V_{T} in the unmedicated state. Inhibition curves are generated by plotting the above data vs the dose (concentration) of IDO1 inhibitor. Inhibition of IDO 1 is then calculated based on the maximal reduction of PET radioligand's V_{T} or BP that can be achieved by a blocking drug at Eₘₐₓ, Tₘₐₓ or Tₘᵢₙ and the change of its non-specific volume of distribution (V_{ND}) and the BP in the presence of IDO1 inhibitor at the various dosing paradigms as compared to the BP or V_{T} in the unmedicated state. The ID50 values are obtained by curve fitting the dose-rate/inhibition curves.

The present invention is further directed to a method for the diagnostic imaging of the IDO1 binding site in a patient which includes the step of combining radiolabeled IDO1 inhibitor with a pharmaceutical carrier or excipient.

### Definitions

Unless otherwise stated, the following terms used in this application, including the specification and claims, have the definitions given below. It must be noted that, as used in the specification and the appended claims, the singular forms “a”, “an” and “the” include plural referents unless the context clearly dictates otherwise. Unless otherwise indicated, conventional methods of mass spectroscopy, NMR, HPLC, protein chemistry, biochemistry, recombinant DNA techniques and pharmacology are employed. In this application, the use of “or” or “and” means “and/or” unless stated otherwise. Furthermore, use of the term “including” as well as other forms, such as “include”, “includes”, and “included”, is not limiting. The section headings used herein are for organizational purposes only and are not to be construed as limiting the subject matter described.

The term “acceptable” with respect to a formulation, composition or ingredient, as used herein, means having no persistent detrimental effect on the general health of the subject being treated.

The term “inhibitor,” as used herein, refers to a molecule such as a compound that binds to a specific binding site on an enzyme and triggers a response in the cell.

The terms “co-administration” or the like, as used herein, are meant to encompass administration of the selected therapeutic agents to a single patient, and are intended to include treatment regimens in which the agents are administered by the same or different route of administration or at the same or different time.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or ignore of the ingredient. Accordingly, the pharmaceutical compositions of the present invention encompass any composition made by mixing a compound of the present invention and a pharmaceutically acceptable carrier. By “pharmaceutically acceptable” it is meant the carrier, diluent or excipient must be compatible with the other ingredients of the formulation and not deleterious to the recipient thereof. The terms “administration of” and or “administering a” compound should be understood to mean providing a compound of the invention or a prodrug of a compound of the invention to the patient.

The terms “effective amount” or “therapeutically effective amount”, as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study.

The term “diagnostically effective” as used herein, means an amount of the imaging composition according to the invention sufficient to achieve the desired effect of concentrating the imaging agent for imaging tissues in a subject as sought by a researcher or a clinician. The amount of an imaging composition of the invention which constitutes a diagnostically effective amount can be determined routinely by one of ordinary skill in the art having regard to their own knowledge, methods known in the art, and this disclosure.

The term “subject” or “patient” encompasses mammals. Examples of mammals include, but are not limited to, humans, chimpanzees, apes, monkey, cattle, horses, sheep, goats, swine, rabbits, dogs, cats, rodents, rats, mice guinea pigs, and the like. In one embodiment, the mammal is a human.

The terms “treat”, “treating” or “treatment”, as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

The compounds herein described may have asymmetric centers. Such compounds containing an asymmetrically substituted atom may be isolated in optically active or racemic forms. It is well known in the art how to prepare optically active forms, such as by resolution of racemic forms or by synthesis from optically active starting materials. Many geometric isomers of olefins, C═N double bonds, and the like can also be present in the compounds described herein, and all such stable isomers are contemplated in the present invention. Cis- and trans-geometric isomers of the compounds disclosed are described and may be isolated as a mixture of isomers or as separated isomeric forms. All chiral, diastereomeric, racemic forms, and all geometric isomeric forms of a structure are intended, unless the specific stereochemistry or isomeric form is specifically indicated.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof.

The terms pharmaceutically acceptable “salt” and “salts” may refer to basic salts formed with inorganic and organic bases. Such salts include ammonium salts; alkali metal salts, such as lithium, sodium, and potassium salts; alkaline earth metal salts, such as calcium and magnesium salts; salts with organic bases, such as amine like salts (e.g., dicyclohexylamine salt, benzathine, N-methyl-D-glucamine, and hydrabamine salts); and salts with amino acids like arginine, lysine, and the like; and zwitterions, the so-called “inner salts”. Nontoxic, pharmaceutically acceptable salts are preferred, although other salts are also useful, e.g., in isolating or purifying the product.

The term pharmaceutically acceptable “salt” and “salts” also includes acid addition salts. These are formed, for example, with strong inorganic acids, such as mineral acids, for example sulfuric acid, phosphoric acid, or a hydrohalic acid such as HCl or HBr, with strong organic carboxylic acids, such as alkanecarboxylic acids of 1 to 4 carbon atoms which are unsubstituted or substituted, for example, by halogen, for example acetic acid, such as saturated or unsaturated dicarboxylic acids, for example oxalic, malonic, succinic, maleic, fumaric, phthalic, or terephthalic acid, such as hydroxycarboxylic acids, for example ascorbic, glycolic, lactic, malic, tartaric, or citric acid, such as amino acids, (for example aspartic or glutamic acid or lysine or arginine), or benzoic acid, or with organic sulfonic acids, such as (C₁-C₄) alkyl or arylsulfonic acids, which are unsubstituted or substituted, for example by halogen, for example methanesulfonic acid or p-toluenesulfonic acid.

The pharmaceutically acceptable salts can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th Edition, p. 1418, Mack Publishing Company, Easton, Pa. (1985), the disclosure of which is hereby incorporated by reference.

Throughout the specification, groups and substituents thereof may be chosen by one skilled in the field to provide stable moieties and compounds and compounds useful as pharmaceutically-acceptable compounds and/or intermediate compounds useful in making pharmaceutically-acceptable compounds.

### EXAMPLES

The synthesis of the compound of the present invention is shown in the following examples.

HPLC Conditions:

Method A: Waters Acquity SDS using the following method: Linear Gradient of 2% to98% solvent B over 1.6 min; UV visualization at 220 nm; Column: BEH C18 2.1 mm×50 mm; 1.7 um particle (Heated to Temp. 50° C.); Flow rate: 1 ml/min; Mobile phase A: 100% Water, 0.05% TFA; Mobile phase B: 100% Acetonitrile, 0.05% TFA.

Method B: Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.00 mL/min; Detection: UV at 220 nm.

### EXAMPLES

### [¹⁸F](R)-N-(4-chlorophenyl)-2-((1S,4S)-4-(6-fluoroquinolin-4-yl)cyclohexyl)propanamide

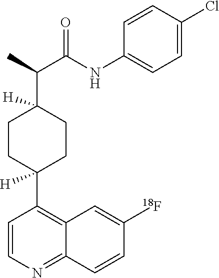 [SMILES: C[C@@H](C(=O)Nc1ccc(Cl)cc1)[C@H]1CC[C@@H](c2ccnc3ccc([18F])cc32)CC1]

### Example 1A

(+/−)-Cis and trans-N-(4-chlorophenyl)-2-(4-(6-iodoquinolin-4-yl)cyclohexyl)propanamide

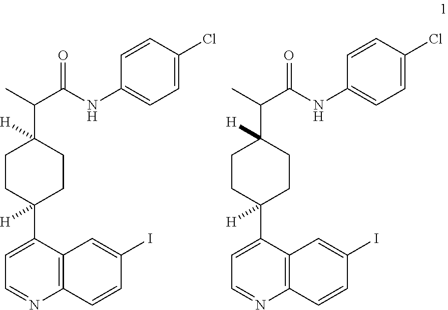 [SMILES: CC(C(=O)Nc1ccc(Cl)cc1)[C@H]1CC[C@@H](c2ccnc3ccc(I)cc32)CC1 ; CC(C(=O)Nc1ccc(Cl)cc1)[C@H]1CC[C@H](c2ccnc3ccc(I)cc32)CC1]

Preparation 1A. Ethyl 2-(1,4-dioxaspiro[4.5]decan-8-ylidene)propanoate

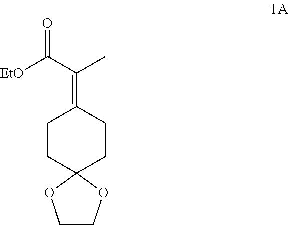 [SMILES: CCOC(=O)C(C)=C1CCC2(CC1)OCCO2]

To a suspension of NaH (0.307 g, 7.68 mmol) in THF (8 mL) cooled at 0° C. was added ethyl 2-(diethoxyphosphoryl)propanoate (1.830 g, 7.68 mmol) slowly. After 30 min, 1,4-dioxaspiro[4.5]decan-8-one (1 g, 6.40 mmol) was added. The resulting mixture was stirred at 0° C. for 2 h, then warmed to rt overnight. The mixture was quenched with water, and THF was removed under reduced pressure. The residue was dissolved in EtOAc, washed with water, brine, dried over Na₂SO₄, filtered, and concentrated. The crude material was purified by ISCO (EtOAc/Hex 0-30%). Fractions containing the product were concentrated to yield Preparation 1A (1.2 g, 78% yield) as a light yellow oil. ¹H NMR (400 MHz, chloroform-d) δ 4.19 (q, J=7.1 Hz, 2H), 4.03-3.89 (m, 4H), 2.68-2.53 (m, 2H), 2.46-2.28 (m, 2H), 1.89 (s, 3H), 1.78-1.66 (m, 4H), 1.30 (t, J=7.1 Hz, 3H).

Preparation 1B. Ethyl 2-(1,4-dioxaspiro[4.5]decan-8-yl)propanoate

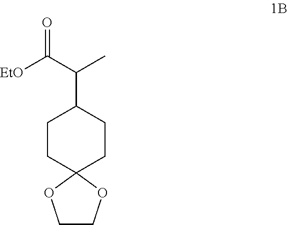 [SMILES: CCOC(=O)C(C)C1CCC2(CC1)OCCO2]

A suspension of Preparation 1A (500 mg, 2.081 mmol) (307A) and 10% palladium on carbon (25 mg, 0.024 mmol) in EtOAc (5 mL) was hydrogenated in a Parr shaker at 45 psi for 6 h. The catalyst was filtered, the filtrate was concentrated to yield Preparation 1B (450 mg, 89% yield) as a light oil. ¹H NMR (400 MHz, chloroform-d) δ 4.12 (dtt, J=10.7, 7.1, 3.6 Hz, 2H), 3.98-3.81 (m, 4H), 2.35-2.17 (m, 1H), 1.83-1.68 (m, 3H), 1.66-1.45 (m, 4H), 1.43-1.28 (m, 2H), 1.27-1.22 (m, 3H), 1.14-1.07 (m, 3H).

Preparation 1C. Ethyl 2-(4-oxocyclohexyl)propanoate

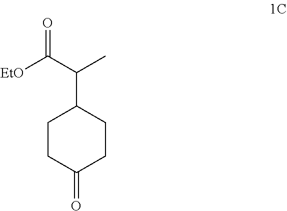

To a solution of Preparation 1B (450 mg, 1.857 mmol) in THF (5 mL) was added 1M hydrogen chloride (aqueous) (0.929 mL, 3.71 mmol). The mixture was heated to 50° C. for 6 h. The reaction mixture was concentrated. The residue was dissolved in EtOAc, washed with water (2×), brine, dried over Na₂SO₄ and concentrated. The crude material was purified with ISCO (EtOAc/Hex 0-30%). Fractions containing product were concentrated to yield Preparation 1C (290 mg, 79% yield) as a clear oil. ¹H NMR (400 MHz, chloroform-d) δ 4.22-4.06 (m, 2H), 2.46-2.30 (m, 5H), 2.13-1.91 (m, 3H), 1.56-1.42 (m, 2H), 1.31-1.24 (m, 3H), 1.18 (d, J=7.1 Hz, 3H).

Preparation 1D. Ethyl 2-(4-(((trifluoromethyl)sulfonyl)oxy)cyclohex-3-en-1-yl)propanoate

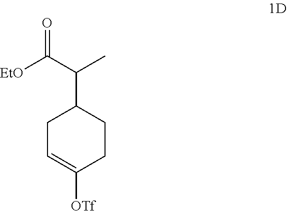 [SMILES: CCOC(=O)C(C)C1CC=C(OS(=O)(=O)C(F)(F)F)CC1]

Preparation 1C (200 mg, 1.01 mmol)(307C) and 2,6-di-tert-butyl-4-methylpyridine (238 mg, 1.16 mmol) were dissolved in dry DCM (10 ml). To the reaction mixture trifluoromethanesulfonic anhydride (0.186 mL, 1.11 mmol) was added dropwise and stirred for 2 h. The suspension was filtered. The filtrate was diluted with DCM, washed with 1N HCl (2×), satd. aq. sodium bicarbonate solution, water, brine, dried over Na₂SO₄, filtered, and concentrated to yield Preparation 1D (320 mg, 96% yield) as a brown oil. ¹H NMR (400 MHz, chloroform-d) δ 5.73 (t, J=6.1 Hz, 1H), 4.28-4.05 (m, 2H), 2.52-2.17 (m, 4H), 2.08-1.79 (m, 3H), 1.49 (dt, J=11.1, 6.6 Hz, 1H), 1.31-1.20 (m, 3H), 1.19-1.04 (m, 3H).

Preparation 1E. Ethyl 2-(4-(4,4,5,5-tetramethyl-1,3,2-dioxaborolan-2-yl)cyclohex-3-en-1-yl)propanoate

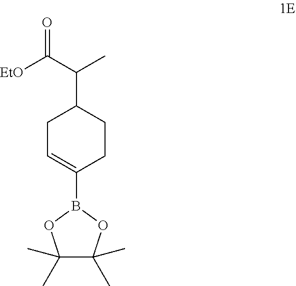 [SMILES: CCOC(=O)C(C)C1CC=C(B2OC(C)(C)C(C)(C)O2)CC1]

To a solution of Preparation 1D (300 mg, 0.908 mmol) (307D) in DMSO (5 mL) was added 4,4,4′,4′,5,5,5′,5′-octamethyl-2,2′-bi(1,3,2-dioxaborolane) (230 mg, 0.908 mmol) and potassium acetate (267 mg, 2.72 mmol). After the mixture was degassed with N₂ for 10 min, PdCl₂(dppf) (19.9 mg, 0.027 mmol) was added. The mixture was heated at 80° C. overnight. The mixture was partitioned between EtOAc and water. The organic phase was concentrated and purified by ISCO. Fractions containing product were concentrated to yield Preparation 1E (168 mg, 60% yield) as a brown oil. ¹H NMR (400 MHz, chloroform-d) δ 6.66-6.40 (m, 1H), 4.31-4.00 (m, 2H), 2.34-2.26 (m, 1H), 2.25-2.19 (m, 1H), 2.19-2.04 (m, 2H), 1.95-1.75 (m, 3H), 1.73-1.60 (m, 1H), 1.29-1.24 (m, 15H), 1.13 (dd, J=11.6, 7.0 Hz, 3H).

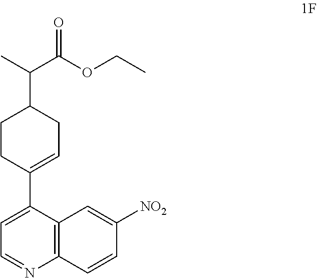 [SMILES: CCOC(=O)C(C)C1CC=C(c2ccnc3ccc([N+](=O)[O-])cc23)CC1]

Preparation 1F. ethyl 2-(4-(6-nitroquinolin-4-yl)cyclohex-3-en-1-yl)propanoate

A 350 mL sealed tube was charged with a mixture of 4-chloro-6-nitroquinoline (2 g, 9.59 mmol), Preparation 1E (3.04 g, 9.88 mmol), Na₂CO₃ (4.06 g, 38.4 mmol), and Pd(Ph₃P)₄ (0.554 g, 0.479 mmol) in dioxane (89 mL) and water (29.6 mL). The reaction was heated at 100° C. overnight. The reaction was quenched with water and diluted with EtOAc. Layers were separated. The aqueous phase was extracted with EtOAc (3×). The organics were combined, dried over Na₂SO₄, filtered, and concentrated to afford a brown residue. Purification of the crude material by silica gel chromatography using an ISCO machine (80 g column, 60 mL/min, 0-45% EtOAc in hexanes over 19 min, tᵣ=14 min) gave Preparation 1F (2.955 g, 8.34 mmol, 87% yield) as a yellow residue. ESI MS (M+H)+=355.2. HPLC Peak tᵣ=0.98 minutes. HPLC conditions: A.

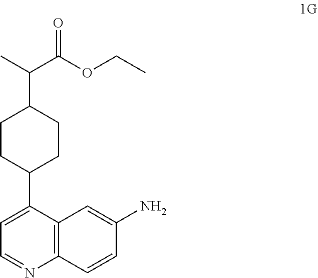 [SMILES: CCOC(=O)C(C)C1CCC(c2ccnc3ccc(N)cc23)CC1]

Preparation 1G. ethyl 2-(4-(6-aminoquinolin-4-yl)cyclohexyl)propanoate

To a solution of Preparation 1F (0.455 g, 1.284 mmol) in MeOH (6.42 ml) was added ammonium formate (0.405 g, 6.42 mmol) followed by Pd/C (0.037 g, 0.347 mmol). The reaction was heated at 70° C. for 1 h. The reaction was filtered through Celite and the filter cake washed with CH₂Cl₂. The filtrate was concentrated. The crude material was taken up in EtOAc and washed with a sat. aq. solution of NaHCO₃ (2×). The organic phase was dried over Na₂SO₄, filtered, and concentrated to afford Preparation 1G (379 mg, 90%) as a brown residue. NMR showed pure desired product in a 1.8:1 dr. ESI MS (M+H)+=327.3. HPLC Peak tᵣ=0.71 minutes. HPLC conditions: A.

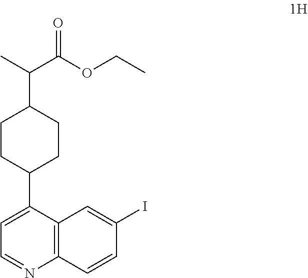 [SMILES: CCOC(=O)C(C)C1CCC(c2ccnc3ccc(I)cc23)CC1]

Preparation 1H. ethyl 2-(4-(6-iodoquinolin-4-yl)cyclohexyl)propanoate

To a solution of Preparation 1G (0.379 g, 1.161 mmol) and aq. HCl (0.59 mL) in water (2.1 mL) was cooled to 0° C., then a solution of sodium nitrite (0.096 g, 1.393 mmol) in water (2.1 mL) was added. A solution of potassium iodide (0.289 g, 1.742 mmol) in water (2.1 mL) was added dropwise to the above solution after the solid dissolved completely. After addition, the mixture was stirred for 30 min at rt, then heated at 70° C. for 1 h. After cooling, the solution was neutralized by slow addition of a solution of Na₂S₂O₃ (1.81 mL), then extracted with CH₂Cl₂ (2×). The organic phase was washed with water, dried over Na₂SO₄, filtered, and concentrated to afford a brown residue. The crude material was dissolved in a minimal amount of CH₂Cl₂ and chromatographed. Purification of the crude material by silica gel chromatography using an ISCO machine (40 g column, 40 mL/min, 0-55% EtOAc in hexanes over 15 min, tᵣ=10.5 min) gave Preparation 1H (92.7 mg, 0.212 mmol, 18.26% yield) as a yellow residue. ESI MS (M+H)+=438.1. HPLC Peak tᵣ=0.89 minutes. HPLC conditions: A.

### Example 1A

### (+/−)-Cis and trans-N-(4-chlorophenyl)-2-(4-(6-iodoquinolin-4-yl)cyclohexyl)propanamide

To a solution of 4-chloroaniline (0.464 g, 3.64 mmol) in THF (2.8 mL) at 0° C. was added a solution of isopropylmagnesium chloride (1.820 mL, 3.64 mmol). The resulting solution was warmed to rt and stirred for 5 min, then Preparation 1H (0.796 g, 1.820 mmol) in THF (4.8 mL) was added dropwise. The reaction was heated at 70° C. for 2 h, then allowed to cool to rt. Additional isopropylmagnesium chloride (1.820 mL, 3.64 mmol) was added. The reaction was heated an additional 2 h. The reaction was quenched with a sat. aq. soln. of NH₄Cl and diluted with EtOAc. Layers were separated. The aqueous phase was extracted with EtOAc (3×). The combined organic phases were dried over Na₂SO₄, filtered, and concentrated to afford a residue. Purification of the crude material by silica gel chromatography using an ISCO machine (80 g column, 60 mL/min, 0-65% EtOAc in hexanes over 35 min, tᵣ=27 min) gave (+/−)-cis-Example 1 (455 mg, 0.702 mmol, 39% yield) and (+/−)-trans-Example 1 (111 mg, 12%). The trans diastereomer elutes first followed by the cis diastereomer. ESI MS (M+H)+=519.1. HPLC Peak tᵣ=0.92 minutes. HPLC conditions: A.

### Example 2

### N-(4-chlorophenyl)-2-(4-(6-iodoquinolin-4-yl)cyclohexyl)propanamide

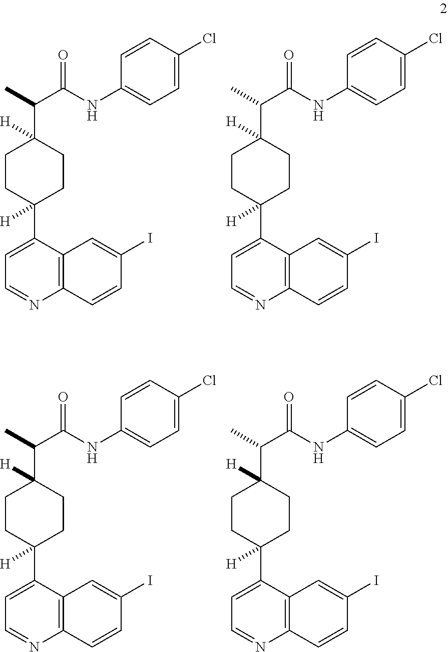 [SMILES: C[C@@H](C(=O)Nc1ccc(Cl)cc1)[C@H]1CC[C@@H](c2ccnc3ccc(I)cc32)CC1 ; C[C@H](C(=O)Nc1ccc(Cl)cc1)[C@H]1CC[C@@H](c2ccnc3ccc(I)cc32)CC1 ; C[C@@H](C(=O)Nc1ccc(Cl)cc1)[C@H]1CC[C@H](c2ccnc3ccc(I)cc32)CC1 ; C[C@H](C(=O)Nc1ccc(Cl)cc1)[C@H]1CC[C@H](c2ccnc3ccc(I)cc32)CC1]

Approximately 65.1 mg of diastereomeric and racemic Example 1 was resolved. The isomeric mixture was purified via preparative SFC with the following conditions: Column: OJ-H, 25×3 cm ID, 5-μm particles; Mobile Phase A: 80/20 CO₂/MeOH; Detector Wavelength: 220 nm; Flow: 150 mL/min. The fractions (“Peak-1” tᵣ=4.64 min, “Peak-2” tᵣ=5.35 min, “Peak-3” tᵣ=6.43 min) were collected in MeOH. The stereoisomeric purity of Peak 1 and 2 were estimated to be greater than 95% based on the prep-SFC chromatograms. Peak 3 was re-purified via preparative SFC with the following conditions to give Isomers 3 and 4: Column: Lux-Cellulose, 25×3 cm ID, 5-μm particles; Mobile Phase A: 75/25 CO₂/MeOH; Detector Wavelength: 220 nm; Flow: 180 mL/min. The fractions (“Peak-1” tᵣ=7.63 min and “Peak-2” tᵣ=8.6 min) was collected in MeOH. The stereoisomeric purity of the fractions was estimated to be greater than 95% based on the prep-SFC chromatograms. Each diasteromer or enantiomer was further purified via preparative LC/MS:

First eluting isomer: The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 50-100% B over 20 minutes, then a 5-minute hold at 100% B; Flow: 20 mL/min. Fractions containing the desired product were combined and dried via centrifugal evaporation to afford Isomer 1 (14.5 mg, 12%). ESI MS (M+H)+=519.2. HPLC Peak tᵣ=2.530 minutes. Purity=92%. HPLC conditions: B. Absolute stereochemistry not determined.

Second eluting isomer: The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 50-100% B over 20 minutes, then a 5-minute hold at 100% B; Flow: 20 mL/min. Fractions containing the desired product were combined and dried via centrifugal evaporation to afford Isomer 2 (8.1 mg, 7.3%). ESI MS (M+H)+=519.1. HPLC Peak tᵣ=2.470 minutes. Purity=100%. HPLC conditions: B. Absolute stereochemistry not determined.

Third eluting isomer: The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 50-100% B over 20 minutes, then a 5-minute hold at 100% B; Flow: 20 mL/min. Fractions containing the desired product were combined and dried via centrifugal evaporation to afford Isomer 3 (13.7 mg, 12%). ESI MS (M+H)+=519.1. HPLC Peak tᵣ=2.481 minutes. Purity=97%. HPLC conditions: B. Absolute stereochemistry not determined.

Fourth eluting isomer: The crude material was purified via preparative LC/MS with the following conditions: Column: XBridge C18, 19×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 50-100% B over 20 minutes, then a 5-minute hold at 100% B; Flow: 20 mL/min. Fractions containing the desired product were combined and dried via centrifugal evaporation to afford Isomer 4 (7.5 mg, 6.7%). ESI MS (M+H)+=518.9. HPLC Peak tᵣ=2.361 minutes. Purity=99%. HPLC conditions: B. Absolute stereochemistry not determined.

### Example 3

### (4-((1S,4S)-4-((R)-1-((4-chlorophenyl)amino)-1-oxopropan-2-yl)cyclohexyl)quinolin-6-yl)diphenylsulfonium trifluoromethanesulfonate

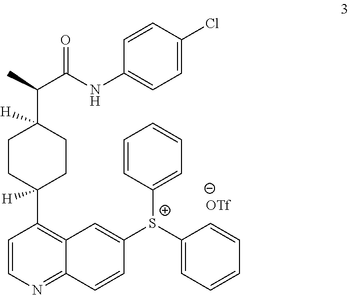 [SMILES: C[C@@H](C(=O)Nc1ccc(Cl)cc1)[C@H]1CC[C@@H](c2ccnc3ccc([S+](c4ccccc4)c4ccccc4)cc32)CC1]

Preparation 3A. (R)-N-(4-chlorophenyl)-2-((1S,4S)-4-(6-(phenylthio)quinolin-4-yl)cyclohexyl)propanamide

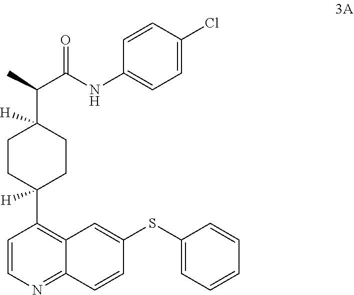 [SMILES: C[C@@H](C(=O)Nc1ccc(Cl)cc1)[C@H]1CC[C@@H](c2ccnc3ccc(Sc4ccccc4)cc32)CC1]

A solution of tris(dibenzylideneacetone)dipalladium(0) (7.9 mg, 0.0086 mmol) and (oxybis(2,1-phenylene))bis(diphenylphosphine) (14 mg, 0.026 mmol) in toluene (1.5 mL) was stirred at ambent temp and degassed by bubbling a low stream of nitrogen through the solution for 5 min. To the solution was added Part X (R)-N-(4-chlorophenyl)-2-((1s,4S)-4-(6-iodoquinolin-4-yl)cyclohexyl)propanamide (90 mg, 0.17 mmol) and the solution was degassed with nitrogen for an additional 3 min. Thiophenol (0.20 mL, 0.21 mmol) and potassium tert-butoxide (23.4 mg, 0.208 mmol) were added and the solution heated at 100° C. for 2 h. The resulting mixture was filtered through a 0.2 pm nylon membrane disc, and loaded onto a 4 gram silica cartridge for purification using an ISCO CombiFlash companion flash system. UV detection was monitored at 254 nm and the flow rate of this purification was 15 mL/min. The normal phase solvents used were; solvent A: hexane, solvent B: ethyl acetate. Using the linear gradient: 0 min-25 min 0% B to 90% B, the purified product eluted between 17 and 24 minutes. Pooled product fractions were evaporated under reduced pressure to give the desired product (R)-N-(4-chlorophenyl)-2-((1S,4S)-4-(6-(phenylthio)quinolin-4-yl)cyclohexyl)propanamide as a yellow solid (intermediate 2) (80 mg, 0.16 mmol). LCMS m/z (M+H,) theory: 501.17, 502.17, 503.17, 504.17 found: 501.34, 502.30, 503.32, 504.34.

(4-((1S,4S)-4-((R)-1-((4-chlorophenyl)amino)-1-oxopropan-2-yl)cyclohexyl)quinolin-6-yl)diphenylsulfonium trifluoromethanesulfonate

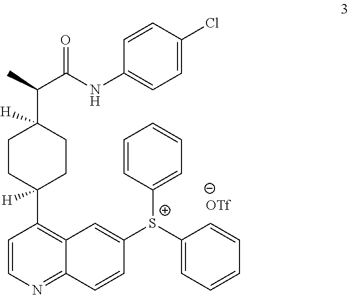 [SMILES: C[C@@H](C(=O)Nc1ccc(Cl)cc1)[C@H]1CC[C@@H](c2ccnc3ccc([S+](c4ccccc4)c4ccccc4)cc32)CC1]

In a 10 mL reaction tube was added a solution of (R)-N-(4-chlorophenyl)-2-((1S,4S)-4-(6-(phenylthio)quinolin-4-yl)cyclohexyl)propanamide from Preparation 3A (80 mg, 0.16 mmol) in Chlorobenzene (1.5 mL). To the solution was added trifluoromethanesulfonic acid (0.028 mL, 0.32 mmol), diphenyliodonium trifluoromethanesulfonate salt (172 mg, 0.399 mmol), and copper benzoate (24.4 mg, 0.080 mmol) and the tube was sealed. The stirred mixture was heated at 125° C. for one hour. After concentration under reduced pressure, the resulting residue was dissolved into acetonitrile (2 mL) and subjected to reverse phase semi-preparative HPLC purification. Purification conditions utilized a flow rate of 18 mL/min with a LUNA C18 21.1×250 mm 5μ LC column with solvents A: 0.1% trifluoroacetic acid in water, solvents B: 0.1% trifluoroacetic acid in acetonitrile and UV detection monitored at 254 nm. Using the gradient: 0 min-12 min, 15% B to 95% B, the purified product eluted at 11.5 minutes. Pooled product fractions were evaporated under reduced pressure and the resulting residue dissolved into methylene chloride (20 mL). This solution was washed sequentially with aqueous 1N sodium hydroxide solution, saturated aqueous sodium trifluoromethanesulfonate solution (5 mL) and water (15 mL). The organic layer was dried over anhydrous magnesium sulfate. Removal of the solvent under reduced pressure afforded the desired product (4-((1S,4S)-4-((R)-1-((4-chlorophenyl)amino)-1-oxopropan-2-yl)cyclohexyl)quinolin-6-yl)diphenylsulfonium trifluoromethanesulfonate as a tan solid (73 mg, 0.090 mmol). ¹H NMR (400 MHz, DMSO-d6) □ 10.09 (br s, 1 H, N—H), 9.13 (d, 1 H, J=4.7 Hz), 8.69 (d, 1 H, J=2.0 Hz), 8.35 (d, 1 H, J=9.1 Hz), 7.99 (dd, 1 H, J=9.1, 2.0 Hz), 7.89 (m, 6 H), 7.80 (m, 4 H), 7.72 (d, 1 H, J=4.7 Hz), 7.65 (d, 2 H, J=8.9 Hz), 7.36 (d, 2 H, J=8.9 Hz), 2.83 (m, 1 H), 1.98-1.82 (m, 5 H), 1.71-1.49 (m, 5 H), 1.14 (d, 3 H, J=6.7 Hz); LCMS m/z (M+H,) theory: 577.21, 578.21, 579.20, 580.21 found: 577.40, 578.40, 579.38, 580.36.

### Example 4

### (R)-N-(4-chlorophenyl)-2-((1S,4S)-4-(6-(4,4,5,5-tetramethyl-1,3,2-dioxaborolan-2-yl)quinolin-4-yl)cyclohexyl)propanamide

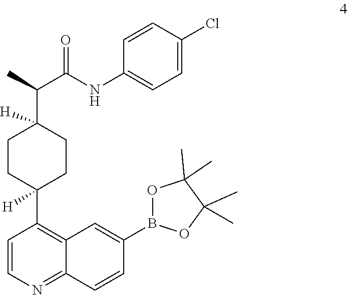 [SMILES: C[C@@H](C(=O)Nc1ccc(Cl)cc1)[C@H]1CC[C@@H](c2ccnc3ccc(B4OC(C)(C)C(C)(C)O4)cc32)CC1]

Preparation 4A. (4-((1S,4s)-4-((R-chlorophenyl)amino)-1-oxopropan-2-yl)cylcohexyl)quinolin-6-yl)boronic Acid

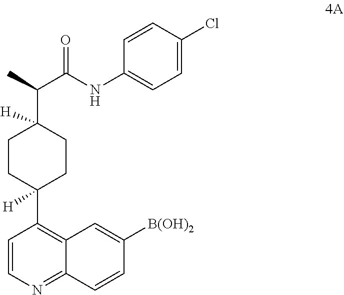 [SMILES: C[C@@H](C(=O)Nc1ccc(Cl)cc1)[C@H]1CC[C@@H](c2ccnc3ccc(B(O)O)cc32)CC1]

(R)-N-(4-chlorophenyl)-2-((1S,4S)-4-(6-iodoquinolin-4-yl)cyclohexyl)propanamide from Example 2 (55 mg, 0.106 mmol) was dissolved in ethanol (5 mL). Tetrahydroxyboron (38 mg, 0.424 mmol, 4 equivalents), 2-(Dicyclohexylphosphino)-2′,4′,6′-Triisopropylbiphenyl (20.2 mg, 0.042 mmol, 0.4 equivalents), potassium acetate (52 mg, 0.530, 5 equivalents) and Chloro(2-dicyclohexylphosphino-2′,4′,6′-Triisopropyl-1,1′-biphenyl)[2-(2′-amino-1,1′-biphenyl)) palladium (II) was added. The reaction mixture was degassed for 5 min, sealed, and heated to 55° C. for 2 hours. The mixture was concentrated and redissolved in acetonitrile/0.1% TFA for preparative HPLC. Purification on a Luna C18(2) column eluting 20 to 90% acetonitrile/0.1% TFA afforded, following collection and pooling of pure fractions and lyophilization, 22.4 mg of (4-((1S,4s)-4-((R-chlorophenyl)amino)-1-oxopropan-2-yl)cylcohexyl)quinolin-6-yl)boronic acid (1A) as a white solid. LC/ms Calculated (M+ 436, 437, 439) Found (M+436.5, 437.5, 439.4).

(R)-N-(4-chlorophenyl)-2-((1s,4S)-4-(6-(4,4,5,5-tetramethyl-1,3,2-dioxaborolan-2-yl)quinolin-4-yl)cyclohexyl)propanamide

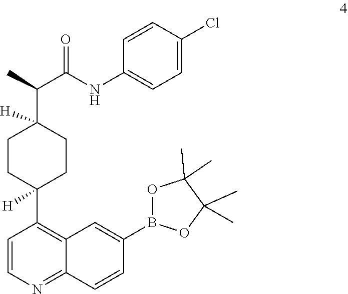 [SMILES: C[C@@H](C(=O)Nc1ccc(Cl)cc1)[C@H]1CC[C@@H](c2ccnc3ccc(B4OC(C)(C)C(C)(C)O4)cc32)CC1]

(4-((1S,4S)-4-((R-chlorophenyl)amino)-1-oxopropan-2-yl)cylcohexyl)quinolin-6-yl)boronic acid (4A) (22.2 mg, 0.051 mmol) was dissolved in 7 mL of methylene chloride. 2,3 dimethylbutane-2,3 diol (7.8 mg, 1.25 equiv) was added along with 12 molecular sieves. The reaction was stirred at room temperature overnight. The reaction was filtered and the filter washed with methylene chloride (2 mL). Concentration afforded 20.4 mg of (R)-N-(4-chlorophenyl)-2-((1s,4S)-4-(6-(4,4,5,5-tetramethyl-1,3,2-dioxaborolan-2-yl)quinolin-4-yl)cyclohexyl)propanamide. LCMS calculated (M+518.0, 519.0, 521) Found (M+518.5, 519.5, 521.5) NMR (CDCl₃, 400 mHz), □□8.9□(d, 1H), 8.65 (s, 1H), 8.1 (m, 2H), 7.6-7.5 (m, 3H), 7.4-7.3 (m, 2H), 3.5 (m, 1H), 2.7 (m, 1H), 2.2-2.1 (m, 1H), 1.9-1.8 (m, 6H), 1.4 (d, CH₃, 12 H), 1.3 (d, CH₃, 3H), 1.3-1.2 (m, 2H).

### Example 5

### [¹⁸F](R)-N-(4-chlorophenyl)-2-((1S, 4S)-4-(6-fluoroquinolin-4-yl)cyclohexyl)propanamide

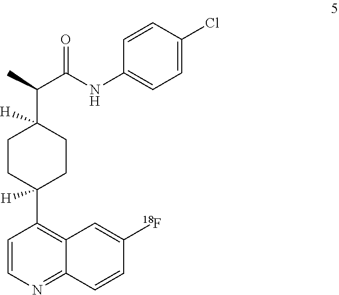 [SMILES: C[C@@H](C(=O)Nc1ccc(Cl)cc1)[C@H]1CC[C@@H](c2ccnc3ccc([18F])cc32)CC1]

### Example 5A

### [¹⁸F](R)-N-(4-chlorophenyl)-2-((1S,4S)-4-(6-fluoroquinolin-4-yl)cyclohexyl)propanamide via Example 3

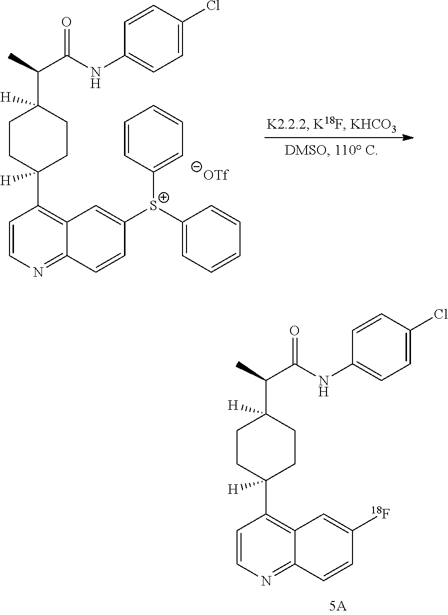 [SMILES: C[C@@H](C(=O)Nc1ccc(Cl)cc1)[C@H]1CC[C@@H](c2ccnc3ccc([S+](c4ccccc4)c4ccccc4)cc32)CC1 ; C[C@@H](C(=O)Nc1ccc(Cl)cc1)[C@H]1CC[C@@H](c2ccnc3ccc([18F])cc32)CC1 ; "OTf"]

An aqueous [¹⁸F]-Fluoride solution (2.0 ml, 28.7 GBq/775 mCi) was purchased from Siemens' PETNET Solutions in Hackensack, N.J. and directly transferred to a QMA Sep-Pak [The Sep-Pak light QMA cartridge (Waters part #186004540) was pre-conditioned sequentially with 5 ml of 8.4% sodium bicarbonate solution, 5 ml of sterile water, and 5 ml of acetonitrile before use] within a custom made remote controlled synthesis unit at BMS in Wallingford, Conn. Upon completion of this transfer, the aqueous [¹⁸F] fluoride was released from the QMA Sep-Pak by the addition of a mixture of 225 mL of an aqueous solution containing 30 mM potassium bicarbonate (4.5 mg, 0.045 mmol) and 30 mM 4,7,13,16,21,24-hexaoxa-1,10-diazabicyclo[8.8.8]hexacosane (17.0 mg, 0.045 mmol) and 1.275 mL of acetonitrile. The solvent was evaporated under a gentle stream of nitrogen at 100° C. and vacuum. Azeotropic drying was repeated twice with 1 ml portions of acetonitrile to generate the anhydrous K.2.2.2/K¹⁸F complex. Upon completion of this process the crypt and was further dried under full vacuum for a 20 minute period. (4-((1S,4S)-4-((R)-1-((4-Chlorophenyl)amino)-1-oxopropan-2-yl)cyclohexyl)quinolin-6-yl)diphenylsulfonium trifluoromethanesulfonate (2.1 mg, 2.8 μmol) was dissolved in anhydrous DMSO (1 mL) and added to the dried cryptand. This solution was heated at 110° C. for 15 minutes. After this time, the crude reaction mixture was diluted with 7 ml of sterile water and 1 mL of acetonitrile. The entire contents were delivered to a Sep-Pak tC18 (400 mg of tC18, 0.8 mL volume, Waters part # WAT036810). The Sep-Pak was rinsed with sterile water (2 mL) to remove unreacted fluoride and then the product was eluted from the Sep-Pak with 2 mL of acetonitrile. The acetonitrile was diluted with sterile water (2 mL) and mixed well. The solution was transferred to the HPLC injection loop and purified by reverse phase HPLC (HPLC Column: Agilent Zorbax SB-C18, 250×10 mm, 5 □m. Solvent A: water with 0.05% TFA. Solvent B: acetonitrile with 0.05% TFA. Conditions: 60% A, 0-15 min; 60-0% A, 15-25 min; 0% A, 25-30 min. Flow 4 mL/min. UV 232 nm. A Gamma ram was used for radiochemical detection.). [¹⁸F](R)-N-(4-Chlorophenyl)-2-((1S,4S)-4-(6-fluoroquinolin-4-yl)cyclohexyl)propanamide was collected over about a 1 min period at 18.1 min in the chromatogram. This product was collected into a 50 ml flask that contained 25 ml of sterile water and the entire contents were delivered to a Sep-Pack light C18 cartridge (130 mg of C18, 0.3 mL volume, Waters part # WAT023501). The Sep-Pak was rinsed with 1 mL of sterile water and the product was released with 0.5 mL of anhydrous ethanol. The ethanol solution of [¹⁸F](R)-N-(4-chlorophenyl)-2-((1S,4S)-4-(6-fluoroquinolin-4-yl)cyclohexyl)propanamide was analyzed by reverse phase HPLC (Column: Agilent Zorbax SB-C18, 250×4.6 mm. 5 □m. Solvent A: water with 0.05% TFA. Solvent B: acetonitrile with 0.05% TFA. Conditions: 58% A, 0-15 min; 58-0% A, 15-25 min; 0% A, 25-30 min. Flow 1 mL/min. UV 232 nm. A Gamma ram was used for radiochemical detection, retention time 13.2 min, the radiochemical purity was 100%. The labeled product co-eluted with an authentic standard of (R)-N-(4-chlorophenyl)-2-((1S,45)-4-(6-fluoroquinolin-4-yl)cyclohexyl)propanamide. The chiral purity was analyzed by reverse phase HPLC (Column: Daicel ChiralCel OD-RH, 150×4.6 mm. 5 □m. Solvent A: water. Solvent B: acetonitrile. Conditions: 40% A, 0-13 min; 40-20% A, 13-14 min; 20% A, 14-16 min; 20-40% A 16-17 min; 40% A 17-20 min. Flow 1 mL/min. UV 232 nm. A Gamma ram was used for radiochemical detection, retention time 7.7 min, the chiral radiochemical purity was 100%. The labeled material gave only one peak when co-injected with an authentic standard of (R,S)-N-(4-chlorophenyl)-2-((1S,4S)-4-(6-fluoroquinolin-4-yl)cyclohexyl)propanamide. The total activity isolated at the end of the synthesis was 4.78 mCi (176.9 MBq) and the specific activity was 7.15 mCi/nmol.

### Example 5B

### [¹⁸F](R)-N-(4-chlorophenyl)-2-((1S,4S)-4-(6-fluoroquinolin-4-yl)cyclohexyl)propanamide via Example 4

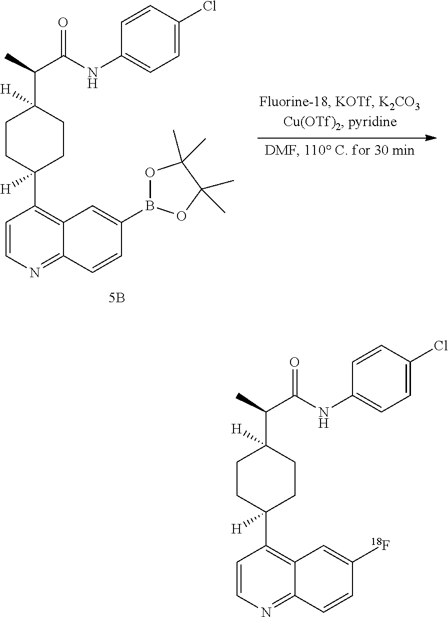 [SMILES: C[C@@H](C(=O)Nc1ccc(Cl)cc1)[C@H]1CC[C@@H](c2ccnc3ccc(B4OC(C)(C)C(C)(C)O4)cc32)CC1 ; C[C@@H](C(=O)Nc1ccc(Cl)cc1)[C@H]1CC[C@@H](c2ccnc3ccc([18F])cc32)CC1]

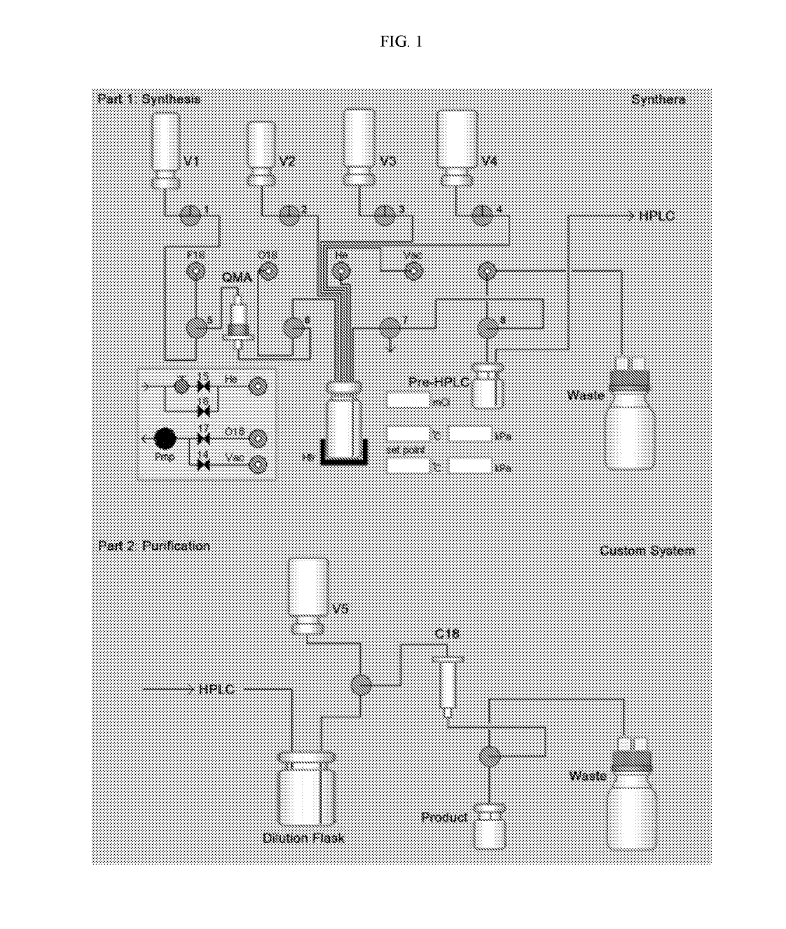

Automated synthesis using commercial Synthera synthesis module (IBA) and custom HPLC system. The automated synthesis of [18F](R)-N-(4-chlorophenyl)-2-((1S,4S)-4-(6-fluoroquinolin-4-yl)cyclohexyl)propanamide was carried out using a cassette type IBA Synthera synthesis module with an appropriately assembled integrator fluidic processor kit for the reaction. Followed by transfer to a custom automated system for HPLC purification and reformulation. The integrator fluidic processor (IFP) kit and custom system were loaded with appropriate precursors for this synthesis and are summarized in Table 1 and a schematic of this system is shown in FIG. 1. Purification was performed on a Varian HPLC unit with filling of the injection loop controlled by a steady stream of nitrogen.

Aqueous [¹⁸F] fluoride solution (2.0 ml, 59.2 GBq/1.6 Ci) was delivered to a Sep-Pak light 46 mg QMA that had been pre-conditioned. After completion of the transfer, aqueous [¹⁸F] fluoride was released from the QMA Sep-Pak by addition of the elution mixture (from “V1”) into the reactor. The solvent was evaporated under a gentle stream of nitrogen and vacuum. Then a solution of precursor (from “V2”) was added to the dried fluoride-18 and heated at 110° C. for 30 minutes. After it was diluted with 2.5 mL of distilled water and 1.5 mL of acetonitrile (from “V4”) followed with transfer to an intermediate vial (to “Pre-HPLC”).

The mixture was then loaded onto a 5 mL sample injection loop then to the semi-preparative HPLC column. A mixture of 40% acetonitrile in an aqueous 0.1% trifluoroacetic acid solution was flushed through the column at a rate of 4.0 mL per minute, pressure 1850 PSI, UV 220 nm. Product was isolated from 22 to 24 min into the dilution flask which contained 30 mL distilled water. The entire contents were transferred to a C18 solid phase extraction cartridge that was pre-activated then released with 1 mL of ethanol (from “V5”) into the product vial of 4 mL saline, to create a 20% ethanol in saline solution for injection. 31.2 mCi (1.15 GBq) of [18F](R)-N-(4-chlorophenyl)-2-((1S,4S)-4-(6-fluoroquinolin-4-yl)cyclohexyl)propanamide.

This product was analyzed via reverse phase HPLC and the chemical identified by co-injection of non-radioactive reference standard of (R)-N-(4-chlorophenyl)-2-((1S,4S)-4-(6-fluoroquinolin-4-yl)cyclohexyl)propanamide, radiochemical purity, chemical purity and specific activity. The isolated product that co-eluted with non-radioactive reference standard at 16 min was 99% radiochemically and 95% chemically pure, with a specific activity of 0.38 GBq/nmol (10.47 mCi/nmol). The product was analyzed via chiral HPLC: chiral purity by co-injection of non-radioactive reference standards (R)-N-(4-chlorophenyl)-2-((1S,4S)-4-(6-fluoroquinolin-4-yl)cyclohexyl)propanamide (10 min) and (S)-N-(4-chlorophenyl)-2-((1S,4S)-4-(6-fluoroquinolin-4-yl)cyclohexyl)propanamide (11.5 min). The isolated product co-eluted with the non-radioactive reference standard at 10 min with an ee: >99.5%.

TABLE 1

| | |
|---|---|
| Vial 1 (V1) | 6 mg potassium trifluoromethanesulfonate |
| | 1.5 mg potassium carbonate |
| | 0.5 mL of distilled water |
| | 1.0 mL of acetonitrile |
| QMA | Sep-Pak Accell Plus QMA Carbonate Plus Light |
| | Cartridge, 46 mg, 40 μM particle (Waters: |
| | PN 186004540) |
| | Pre-conditioned with: |
| | 1) 10 mL ethanol |
| | 2) 900 mg potassium trifluoromethanesulfonate |
| | in 10 mL distilled water |
| | 3) 10 mL of distilled water |
| Vial 2 (V2) | 2 mg (R)-N-(4-chlorophenyl)-2-((1S,4S)-4-(6-(4,4,5,5- |
| | tetramethyl-1,3,2-dioxaborolan-2-yl)quinolin-4- |
| | yl)cyclohexyl)propanamide |
| | 7 mg Copper(II) trifluoromethanesulfonate |
| | 40 μL pyridine |
| | 0.7 mL N,N-Dimethylformamide |
| Vial 4 (V4) | 2.5 mL of distilled water |
| | 1.5 mL acetonitrile |
| HPLC Column | Phenomenex Luna, 5 μm C18(2) 100 Å, |
| | 250 × 10 mm (PN 00G-4252-N0) |
| HPLC Solvent | 40% acetonitrile in an aqueous 0.1% trifluoroacetitic |
| | acid solution |
| HPLC flow | 4.0 mL/min |
| Dilution Flask | 30 mL of distilled water |
| Cartridge | Phenomenex Strata C18-U (55 μM, 70 Å), |
| | 100 mg/1 mL Tube (PN 8B-S002-EAK) |
| | Pre-conditioned with: |
| | 5 mL ethanol |
| | 2) 10 mL distilled water |
| Vial 5 (V5) | 1 mL ethanol |
| Product Vial | 4 mL saline |

### Example 6

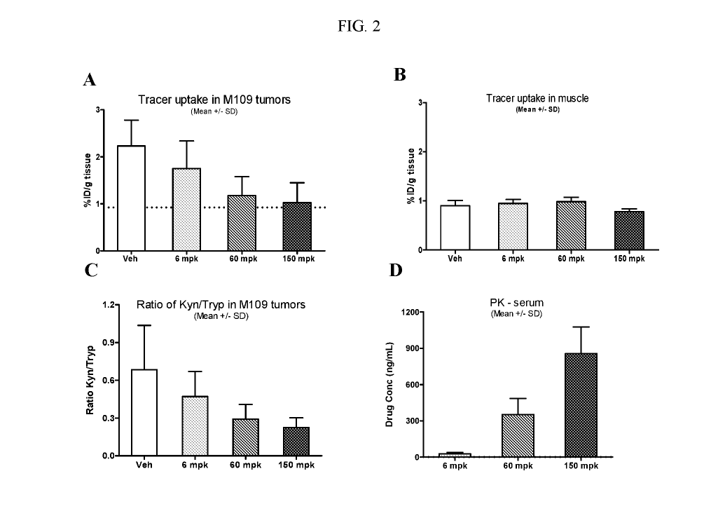

### In-vivo PET Imaging with [¹⁸F](R)-N-(4-chlorophenyl)-2-((1S,4S)-4-(6-fluoroquinolin-4-yl)cyclohexyl)propanamide in IDO1 Expressing Xenograft Mouse Model

[¹⁸F](R)-N-(4-chlorophenyl)-2-((1S,4S)-4-(6-fluoroquinolin-4-yl)cyclohexyl)propanamide was tested to confirm its properties as an IDO1 PET radioligand. [¹⁸F](R)-N-(4-chlorophenyl)-2-((1S,4S)-4-(6-fluoroquinolin-4-yl)cyclohexyl)propanamide was tested for its specificity and targeting for the IDO1 enzyme using PET imaging of M109 mouse tumor models. The M109 tumor model is generated from a murine lung carcinoma cell line and expresses high levels of IDO1. Xenograft tumor models were generated by implanting 1×10⁶ M109 cells subcutaneously on the right shoulder of BALB/c mice. After the implant, the tumors were allowed to grow for 5 days, before the studies began. 45 mice with implanted M109 xenografts were divided into 4 groups. In Group 1, 12 animals received 6 mg/kg (R)-N-(4-chlorophenyl)-2-((1S,4S)-4-(6-fluoroquinolin-4-yl)cyclohexyl)propanamide (n=12), in Group 2, 12 animals received 60 mg/kg (R)-N-(4-chlorophenyl)-2-((1S,4S)-4-(6-fluoroquinolin-4-yl)cyclohexyl)propanamide (n=12), in Group 3, 11 animals received 150 mg/kg (R)-N-(4-chlorophenyl)-2-((1S,4S)-4-(6-fluoroquinolin-4-yl)cyclohexyl)propanamide (n=11) and in Group 4, 10 animals received a vehicle of saline (n=10). Dosing and treatment was established based on known pharmacological effect and treatment was administered PO, once daily, for 4 or 5 days. All animals were received a PET scan post-treatment with the last dose of (R)-N-(4-chlorophenyl)-2-((1S,4S)-4-(6-fluoroquinolin-4-yl)cyclohexyl)propanamide or vehicle administered 2 hours before PET imaging. 150 μCi of a 10% solution of ethanol in sterile saline for injection containing rF1(R)-N-(4-chlorophenyl)-2-((1S,4S)-4-(6-fluoroquinolin-4-yl)cyclohexyl)propanamide, was i.v. injected 1 hour prior to PET imaging to allow for tracer distribution and uptake in the tumor. The exact injected dose was calculated by subtracting the decay corrected activity of the residual in the syringe after injection from the total measured dose in the syringe prior to injection. For PET imaging, the mice were anesthetized with isoflurane and placed into a custom animal holder with capacity for 4 animals. Body temperature was maintained with a heating pad and anesthesia was maintained with 1.5% isoflurane for the duration of the imaging. PET imaging was performed on a dedicated microPET® F120™ scanner and a F220™ scanner (Siemens Preclinical Solutions, Knoxville, Tenn.). A 10 minute transmission scan was performed using a ⁵⁷Co source for attenuation correction of the final PET images and followed by a 10 min static emission scan. Either before or after PET imaging a CT scan (X-SPECT, Gamma Medica) or MRI scan (Bruker) was performed for anatomical orientation during image analysis. PET images were reconstructed using a maximum a posteriori (MAP) algorithm with attenuation correction using the collected transmission images. Image analysis was performed using the image analysis software AMIDE. PET images were co-registered with their corresponding CT or MRI images and regions of interest (ROIs) were manually drawn around tumor boundaries and muscle using the CT or MRI images as the anatomical guidelines. The outcome measure percentage injected dose/g tissue (% ID/g) was obtained from the ROIs volume and the calculated injected activity decay corrected to the beginning of the emission scan. Tracer uptake in tumors from the (R)-N-(4-chlorophenyl)-2-((1S,4S)-4-(6-fluoroquinolin-4-yl)cyclohexyl)propanamide treated groups were compared to that of the vehicle groups and muscle tissue. Muscle tissue was used as a reference region to evaluate non-specific binding since the IDO1 expression in that tissue was small. In groups 1-3, which were treated with (R)-N-(4-chlorophenyl)-2-((1S,4S)-4-(6-fluoroquinolin-4-yl)cyclohexyl)propanamide (6-150 mg/kg) prior to imaging produced a dose-dependent decrease in tracer uptake compared to the vehicle group (FIG. 2A). In the muscle reference tissue, treatment had no effect on the tracer, consistent with the absence of IDO1 expression in these tissues (FIG. 2B). In a subset of animals, (Veh; n=7, 6 mg/kg; n=7, 60 mg/kg; n=8, 150 mg/kg; n=8) the tumor and serum were collected for measurement of drug levels of (R)-N-(4-chlorophenyl)-2-((1S,4S)-4-(6-fluoroquinolin-4-yl)cyclohexyl)propanamide, tryptophan and kynurenine within the serum and tumor. For these studies, the mice received one additional dose of (R)-N-(4-chlorophenyl)-2-((1S,4S)-4-(6-fluoroquinolin-4-yl)cyclohexyl)propanamide or vehicle the day after imaging. Seven hours following last dose of (R)-N-(4-chlorophenyl)-2-((1S,4S)-4-(6-fluoroquinolin-4-yl)cyclohexyl)propanamide, the mice were euthanized and the tumor and serum was collected and processed for the markers. As shown in FIG. 2A, there was a correlation between tracer signal within the M109 tumors and concentration of (R)-N-(4-chlorophenyl)-2-((1S,4S)-4-(6-fluoroquinolin-4-yl)cyclohexyl)propanamide. As the serum concentration of (R)-N-(4-chlorophenyl)-2-((1S,4S)-4-(6-fluoroquinolin-4-yl)cyclohexyl)propanamide (FIG. 2D) increased with increasing administered dose, the % ID/g within the tumor decreased in a dose dependent manner. The inhibition of the kynurenine pathway, as measured by the ratio of kynurenine to tryptophan (Kyn/Trp) within the tumors is shown in FIG. 2C. A dose-dependent decrease in the ratio of kynurenine to tryptophan was observed in the tumors within the groups treated with (R)-N-(4 -chlorophenyl)-2-((1S,4S)-4-(6-fluoroquinolin-4-y0cyclohexyl)propanamide as compared to the vehicle group and followed the same trend as the % ID/g measured from the PET imaging data. These results provide evidence for specificity and targeting of IDO1 by [¹⁸F](R)-N-(4-chlorophenyl)-2-((1S,4S)-4-(6-fluoroquinolin-4-yl)cyclohexyl)propanamide in vivo, as well as demonstrating the utility of [¹⁸F](R)-N-(4-chlorophenyl)-2-((1S,4S)-4-(6-fluoroquinolin-4-yl)cyclohexyl)propanamide as a PET radioligand for this target. We demonstrated displacement of the tracer by (R)-N-(4-chlorophenyl)-2-((1S,4S)-4-(6-fluoroquinolin-4-yl)cyclohexyl)propanamide in a dose dependent manner in IDO1 expressing M109 tumors. Moreover, our imaging results correlated with a dose-dependent PD effect in the tumors and PK in serum, thus confirming both specificity and target engagement of [¹⁸F](R)-N-(4-chlorophenyl)-2-((1S,4S)-4-(6-fluoroquinolin-4-yl)cyclohexyl)propanamide in vivo.

### Example 7

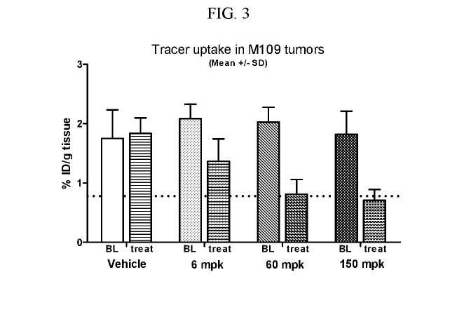

### In-vivo PET Imaging with [¹⁸F](R)-N-(4-chlorophenyl)-2-((1S,4S)-4-(6-fluoroquinolin-4-yl)cyclohexyl)propanamide IDO1 Expressing Xenograft Mouse Model at Baseline and After Treatment with an IDO1 Inhibitor

[¹⁸F](R)-N-(4-chlorophenyl)-2-((1S,4S)-4-(6-fluoroquinolin-4-yl)cyclohexyl)propanamide was tested within a M109 mouse tumor model at baseline and after treatment with an IDO1 inhibitor, (R)-N-(4-chlorophenyl)-2-((1S,4S)-4-(6-fluoroquinolin-4-yl)cyclohexyl)propanamide. The M109 tumor model is generated from a murine lung carcinoma cell line and expresses high levels of IDO1. Xenograft tumor models were generated by implanting 1×10⁶ M109 cells subcutaneously on the right shoulder of BALB/c mice. After the implant, the tumors were allowed to grow for 5 days, before the studies began. 16 mice with implanted M109 xenografts were divided into 4 groups. In Group 1, 4 animals received 6 mg/kg (R)-N-(4-chlorophenyl)-2-((1S,4S)-4-(6-fluoroquinolin-4-yl)cyclohexyl)propanamide (n=12), in Group 2, 4 animals received 60 mg/kg (R)-N-(4-chlorophenyl)-2-((1S,4S)-4-(6-fluoroquinolin-4-yl)cyclohexyl)propanamide (n=12), in Group 3, 4 animals received 150 mg/kg (R)-N-(4-chlorophenyl)-2-((1S,4S)-4-(6-fluoroquinolin-4-yl)cyclohexyl)propanamide (n=11) and in Group 4, 4 animals received a vehicle of saline (n=10). Dosing and treatment was established based on known pharmacological effect and treatment was administered PO, once daily, for 4 or 5 days. All mice underwent 2 separate PET scans. The first was a baseline PET scan before treatment began and the second was a post treatment scan with either the IDO1 inhibitor or vehicle. Treatment was administered PO once daily for 5 days with the last dose administered 2 hours prior to the post-treatment PET scan. 150 μCi of a 10% solution of ethanol in sterile saline for injection containing [¹⁸F](R)-N-(4-chlorophenyl)-2-((1S,4S)-4-(6-fluoroquinolin-4-yl)cyclohexyl)propanamide, was i.v. injected 1 hour prior to PET imaging to allow for tracer distribution and uptake in the tumor. The exact injected dose was calculated by subtracting the decay corrected activity of the residual in the syringe after injection from the total measured dose in the syringe prior to injection. For PET imaging, the mice were anesthetized with isoflurane and placed into a custom animal holder with capacity for 4 animals. Body temperature was maintained with a heating pad and anesthesia was maintained with 1.5% isoflurane for the duration of the imaging. PET imaging was performed on a dedicated microPET® F120™ scanner and a F220™ scanner (Siemens Preclinical Solutions, Knoxville, Tenn.). A 10 minute transmission scan was performed using a ⁵⁷Co source for attenuation correction of the final PET images and followed by a 10 min static emission scan. Either before or after PET imaging a CT scan (X-SPECT, Gamma Medica) or MRI scan (Bruker) was performed for anatomical orientation during image analysis. PET images were reconstructed using a maximum a posteriori (MAP) algorithm with attenuation correction using the collected transmission images. Image analysis was performed using the image analysis software AMIDE. PET images were co-registered with their corresponding CT or MRI images and regions of interest (ROIs) were manually drawn around tumor boundaries and muscle using the CT or MRI images as the anatomical guidelines. The outcome measure percentage injected dose/g tissue (% ID/g) was obtained from the ROIs volume and the calculated injected activity decay corrected to the beginning of the emission scan. Tracer uptake in tumors from the (R)-N-(4-chlorophenyl)-2-((1S,4S)-4-(6-fluoroquinolin-4-yl)cyclohexyl)propanamide treated groups were compared to that of the vehicle groups and muscle tissue. Muscle tissue was used as a reference region to evaluate non-specific binding since the IDO1 expression in that tissue was small. There were no difference in tracer uptake at baseline in the M109 tumors between any of the groups. As shown in FIG. 3, a dose-dependent decrease in tracer uptake was observed in the (R)-N-(4-chlorophenyl)-2-((1S,4S)-4-(6-fluoroquinolin-4-yl)cyclohexyl)propanamide (6-150 mg/kg) treated groups compared to the vehicle group. The tracer uptake did not change between baseline and post-treatment imaging in the vehicle group. Tracer uptake in muscle reference tissue was unaffected by treatment and did not differ between baseline and post-treatment for any of the groups. Combined, these results confirm the targeting of IDO1 by [¹⁸F](R)-N-(4-chlorophenyl)-2-((1S,4S)-4-(6-fluoroquinolin-4-yl)cyclohexyl)propanamide in vivo, as well as, demonstrating feasibility of the baseline post-treatment study design that are used in the clinically to evaluate the correlation between drug exposure/in-vivo target occupancy and efficacy of a therapeutic drug.

### Example 8

### Comparison of Tracer Uptake in a High and Low IDO1 Expressing Tumor Model

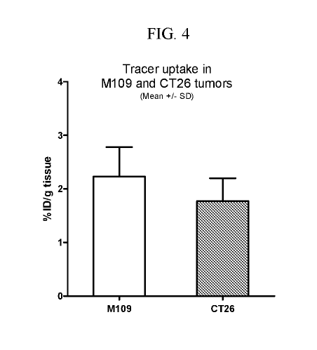

In order to compare the ability to differentiate between differences in expression levels of IDO1 we imaged an additional mouse model carrying CT26 tumors. The CT26 tumor model is generated from a murine colorectal carcinoma cell line and expresses lower levels of IDO1 than the M109 model. Xenograft tumor models were generated by implanting 1×10⁶ CT26 cells subcutaneously on the right shoulder of BALB/c mice. After the implant the tumors were allowed to grow for 7 days, before the studies began. The mice received a vehicle dose for 5 days prior to imaging and the dosing and imaging was performed exactly as described in example 6 to ensure the M109 and CT26 studies were comparable. As shown in FIG. 4, higher % ID/g was observed in M109 mouse xenograft tumors compared to % ID/g in CT26mouse xenograft tumors, consistent with the level of expression of IDO1 respectively in these models.

### Example 9

### Biodistribution in Non-Human Primate

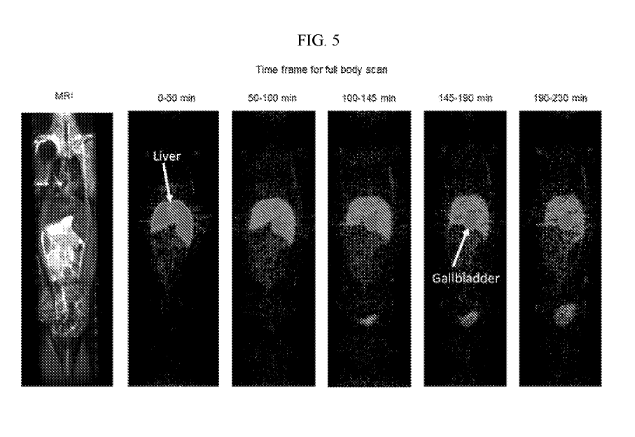

A PET imaging study was performed in a cynomolgus monkey to evaluate the biodistribution and background signal of [¹⁸F](R)-N-(4-chlorophenyl)-2-((1S,4S)-4-(6-fluoroquinolin-4-yl)cyclohexyl)propanamide in a non-human primate. A male cynomolgus monkey (3.5 kg) was anesthetized via a cocktail of 0.02 mg/kg Atropine, and 5 mg/kg Telazol, 0.01 mg/kg Buprenorphine and maintained with 1-2% isoflurane for the duration of the study. Body temperature was maintained at ˜37° C. using an external circulating water bed to prevent hypothermia during imaging. The monkey was intubated and a saphenous vein catheter was inserted to allow for radiotracer injection. 1.2 mCi of a 10% ethanol in sterile saline for in injection containing [¹⁸F](R)-N-(4-chlorophenyl)-2-((1S,4S)-4-(6-fluoroquinolin-4-yl)cyclohexyl)propanamide was i.v. injected and the monkey was placed in a custom made animal holder, compatible with both the MRI and the PET scanner (F220™ scanner, Siemens Preclinical Solutions, Knoxville, Tenn.). The monkey was placed in the MRI scanner for anatomical imaging. Three high-resolution MRI axial images was acquired for full body coverage starting from the head and ending at the hind legs. Following MRI, the monkey was transferred to the PET scanner. The axial field of view in the PET system is 7.6 cm. With this limitation, images were acquired over 7 distinct bed positions to cover the full body with an overlap of 1.5 cm between beds. Prior to emission imaging a 10 minute transmission image was acquired for each bed position for attenuation correction of the final PET images. Immediately following collection of the final transmission image, the bed was returned to position 1 and the radiotracer was injected via the saphenous vein catheter concurrently with the start of the first emission scan. For emission, a total of 5 full body scans were acquired, using 5 min emission acquisitions for each bed position. Images were reconstructed using a MAP algorithm with attenuation correction. Bed positions were stitched together for a full body image using a stitching software tool developed in house and PET and MRI images were co-registered using AMIDE software. The final images were visually inspected to note areas of high tracer accumulation and evaluate biodistribution and background signal. The tracer accumulated in the liver and gallbaldder, consistent with the expected route of excretion. No other areas showed notable accumulation of [¹⁸F](R)-N-(4-chlorophenyl)-2-((1S,4S)-4-(6-fluoroquinolin-4-yl)cyclohexyl)propanamide and the background signal was minimal, as shown in FIG. 5. This result demonstrates a low background signal was detected in non-human primate and should generate high signal/noise ratios where IDO1 expression is increased within the tumor microenvironment for human imaging.

1. A radiolabeled compound having the following Formula I: 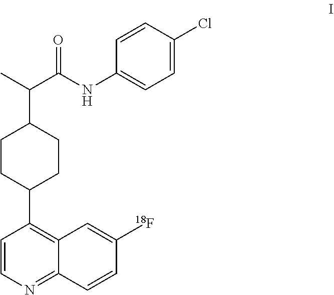 [SMILES: CC(C(=O)Nc1ccc(Cl)cc1)C1CCC(c2ccnc3ccc([18F])cc23)CC1]
or a pharmaceutically acceptable salt thereof.

2. The radiolabeled compound of claim 1 having the following structure: 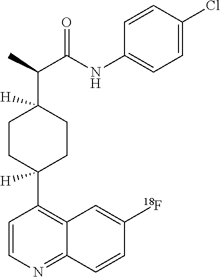 [SMILES: C[C@@H](C(=O)Nc1ccc(Cl)cc1)[C@H]1CC[C@@H](c2ccnc3ccc([18F])cc32)CC1]
or a pharmaceutically acceptable salt thereof.

3. A pharmaceutical composition comprising a diagnostically effective amount of the radiolabeled compound of claim 2 and a pharmaceutically acceptable carrier therefor.

4. A method of in vivo imaging of mammalian tissues of known IDO1 expression to detect cancer cells comprising the steps of:
(a) administering the radiolabeled compound of claim 2 to a subject; and
(b) imaging in vivo the distribution of the radiolabeled compound by positron emission tomography (PET) scanning.

5. A method for screening a non-radiolabeled compound to determine its affinity for occupying the binding site of the IDO1 enzyme in mammalian tissue comprising the steps of:
(a) administering the radiolabeled compound of claim 2 to a subject;
(b) imaging in vivo tissues of known IDO1 expression by positron emission tomography (PET) to determine a baseline uptake of the radiolabeled compound;
(c) administering the non-radiolabeled compound to said subject;
(d) administering a second dose of the radiolabeled compound of claim 2 to said subject;
(e) imaging in vivo the distribution of the radiolabeled compound of claim 2 in tissues that express IDO1 enzymes;
(f) comparing the signal from PET scan data at baseline within the tissue that expresses IDO1 to PET scan data retrieved after administering the non-radiolabeled compound within the tissue that expresses IDO1 enzymes.

6. A method for monitoring the treatment of a cancer patient who is being treated with an IDO1 inhibitor comprising the steps of:
(a) administering to the patient the radiolabeled compound of claim 2;
(b) obtaining an image of tissues in the patient that express IDO1 enzymes by positron emission tomography (PET); and
(c) detecting to what degree said radiolabeled IDO1 inhibitor occupies the binding site of the IDO1 enzyme.

7. A method for tissue imaging comprising the steps of contacting a tissue that contains IDO1 enzymes with the radiolabeled compound of claim 2 and detecting the radiolabeled compound using positron emission tomography (PET) imaging.

8. The method of claim 7 wherein the compound is detected in vitro.

9. The method of claim 7 wherein the compound is detected in vivo.

10. A method for diagnosing the presence of a disease in a subject, comprising
(a) administering to a subject in need thereof the radiolabeled compound of claim 2 which binds to the IDO1 enzyme associated with the presence of the disease; and
(b) obtaining a radio-image of at least a portion of the subject to detect the presence or absence of the radiolabeled compound; wherein the presence and location of the radiolabeled compound above background is indicative of the presence or absence of the disease.

11. A method for quantifying diseased cells or tissues in a subject, comprising
(a) administering to a subject having diseased cells or tissues the radiolabeled compound of claim 2 which binds to the IDO1 enzyme located within the diseased cells or tissues; and
(b) detecting radioactive emissions of the radiolabeled compound in the diseased cells or tissues, wherein the level and distribution of the radioactive emissions in the diseased cells or tissues is a quantitative measure of the diseased cells or tissues.
